(19) United States
(12) Patent Application Publication
(10) Pub. No.: US 2024/0139136 A1
(43) Pub. Date: May 2, 2024

(54) METHODS AND FORMULATIONS FOR TOPICAL ADMINISTRATION OF GABAPENTINOIDS

(71) Applicant: VYLUMA INC., Bridgewater, NJ (US)

(72) Inventors: Jack Martin LIPMAN, West Milford, NJ (US); Kumaresh SOPPIMATH, Skillman, NJ (US); Tushar HINGORANI, Bridgewater, NJ (US)

(21) Appl. No.: 18/547,929

(22) PCT Filed: Feb. 25, 2022
(86) PCT No.: PCT/US2022/017997

§ 371 Date: Aug. 25, 2023

Related U.S. Application Data
(60) Provisional application No. 63/154,623, filed on Feb. 26, 2021.

(51) Int. Cl. A61K 31/197; A61K 9/00; A61K 31/195; A61K 47/02; A61K 47/18; A61K 47/38

(52) U.S. Cl. CPC A61K 31/197; A61K 9/0048; A61K 31/195; A61K 47/02; A61K 47/186; A61K 47/38

(57) ABSTRACT

The present disclosure is directed to novel ophthalmic formulations comprising a gabapentinoid and methods for treating corneal pain using said formulations.

### FIELD AND BACKGROUND OF THE INVENTION

The present disclosure relates to ophthalmically useful pharmaceutical compositions comprising gabapentinoids. In some aspects, the composition is an ophthalmic formulation comprising pregabalin and appropriate pharmaceutical excipients suitable for topical administration to the eye of a subject. The present disclosure also relates to methods for treating corneal pain in a subject in need thereof.

A subject may experience corneal pain for a variety of reasons, such as corneal nerve damage, LASIK surgery, cataract surgery, or other eye surgeries, chronic dry eye disease, radiation therapy, extended contact lens wear, toxicity from preservatives, shingles, diabetes, trigeminal neuralgia, abnormal healing, corneal abrasions, physical or chemical injury, or any systemic condition that can lead to nerve damage.

Anticonvulsant agents such as gabapentinoids have been used to treat pain in patients, but often result in numerous side effects when administered orally. A topical ophthalmic formulation could relieve corneal pain while avoiding negative side effects, as such administration typically leads to lower systemic exposure of the administered drug.

Thus, there is a need to develop topical pharmaceutical compositions capable of treating corneal pain while minimizing side effects.

### BRIEF SUMMARY OF THE INVENTION

### Pharmaceutical Compositions

The present disclosure provides pharmaceutically acceptable ophthalmic formulations comprising gabapentinoids and one or more pharmaceutically acceptable excipients.

In some aspects, the ophthalmic formulation comprises a gabapentinoid present at a concentration of from about 0.1% to about 6.5 wt % and one or more pharmaceutically acceptable excipients.

In some aspects, the gabapentinoid is pregabalin or gabapentin.

In some aspects, the formulation has a viscosity of from about 1 to about 300 cPs and a pH from about 4 to about 8.

In some aspects, the concentration of the gabapentinoid is from about 1 wt % to about 5 wt %.

In some aspects, the concentration of the gabapentinoid is from about 2 wt % to about 4 wt %.

In some aspects, the concentration of the gabapentinoid is about 2 wt %.

In some aspects, the concentration of the gabapentinoid is about 3 wt %.

In some aspects, the concentration of the gabapentinoid is about 4 wt %.

In some aspects, the formulation has a viscosity of from about 5 to about 50 cPs.

In some aspects, the formulation has a pH from about 6.5 to about 7.5.

In some aspects, the formulation is an aqueous solution.

In some aspects, the formulation is a suspension.

In some aspects, the formulation is a gel.

In some aspects, the formulation comprises one or more pharmaceutically acceptable excipients are selected from the group consisting of preservatives, buffers, penetration enhancers, viscosity agents, tonicity regulators, chelating agents, polymers, lipids, ointment bases, and resuspension components, or combinations thereof.

In some aspects, the formulation further comprises an antimicrobial agent.

In some aspects, the formulation has an osmolality of from about 260 to about 365 mOsm/kg.

In some aspects, the formulation is isotonic.

In some aspects, the formulation is hypotonic.

In some aspects, the formulation is hypertonic.

In some aspects, formulation is packaged in an aseptic manner.

In some aspects, the formulation is packaged in a single-dose container.

In some aspects, the formulation is packaged in a multiple-dose container.

### Methods of Treatment

The present disclosure also provides methods of treating corneal pain in a subject, the method comprising administering to the subject a therapeutically effective amount of an ophthalmic formulation comprising a gabapentinoid and one or more pharmaceutically acceptable excipients.

In some aspects, the gabapentinoid is pregabalin or gabapentin.

In some aspects, the corneal pain is acute corneal pain.

In some aspects, wherein the corneal pain is chronic corneal pain.

In some aspects, the subject is a human.

In some aspects, the composition is co-administered with a second pharmaceutical composition.

In some aspects, the second pharmaceutical composition contains an active agent selected from the group consisting of non-steroidal anti-inflammatory drugs, local anesthetics, antimicrobial agents, immune suppressants, and steroids.

In some aspects, the ocular bioavailability of the gabapentinoid is improved by from about 5% to about 50% compared to oral administration of the same gabapentinoid.

In some aspects, the administering is into the cornea, the lower eyelid, an eye surface, or an ophthalmic tissue of at least one eye of the subject.

In some aspects, the administering is performed by intravitreal or intraocular injection.

In some aspects, the intensity of corneal pain in the subject decreases by from about 5 to about 50% as measured by an ocular pain questionnaire compared to treatment with oral administration of the same gabapentinoid or no treatment.

In some aspects, the ocular pain questionnaire is selected from the group consisting of Ocular Pain Assessment Survey, visual analog scale (VAS), numerical rating scale (NRS), Wong-Baker FACES® Pain Rating Scale, and Eye Sensation Scale, or combination thereof.

In some aspects, the subject has reduced side effects compared to oral administration of the same gabapentinoid, wherein the side effects are selected from the group consisting of angioedema, hypersensitivity reactions, suicidal thoughts or behavior, respiratory depression, dizziness, somnolence, peripheral edema, blurred vision, weight gain, and difficulty with concentration and attention, or combinations thereof.

### BRIEF DESCRIPTION OF THE FIGURE

FIG. 1 is a bar graph demonstrating the effect of ophthalmic formulations on the number of ipsilateral paw wipes by rats with post-surgery injury to cornea immediately following treatment with capsaicin. For balanced salt solution (BSS, negative control), 0.5% proparacaine (positive control), 3% nalbuphine, 3% pregabalin, and 3% ketamine, the plot shows the number of eye wipes after i) treatment with artificial tears (Baseline (Pre-Induction)), ii) challenge with capsaicin alone (Baseline), and, iii) challenge with capsaicin 15 minutes after administration of an ophthalmic formulation (15 Minutes).

### DETAILED DESCRIPTION OF THE INVENTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition. The excipient can be an inert substance, an inactive substance, and/or a not medicinally active substance.

The terms “effective amount” or “pharmaceutically effective amount” or “therapeutically effective amount” as used herein refer to the amount or quantity of a drug or pharmaceutically active substance which is sufficient to elicit the required or desired therapeutic response, or in other words, the amount which is sufficient to elicit an appreciable biological response when administered to a patient.

The term “essentially free” means that only trace amounts of other substances, or a reference substance (such trace amounts not having a substantial effect in the application), can be detected.

“Administration”, or “to administer” means the step of giving (i.e. providing) a pharmaceutical composition to a subject. The pharmaceutical compositions disclosed herein can be “locally administered”, that is administered at or in the vicinity of the site at which a therapeutic result or outcome is desired. For example to treat an ocular condition such as corneal pain, topical administration, directly to the eye of a subject, of an ophthalmic formulation can be carried out, and is an example of local administration.

The term “pharmaceutically acceptable salts” is art-recognized, and includes relatively non-toxic, inorganic and organic acid addition salts of compounds and relatively non-toxic, inorganic and organic base addition salts of compounds.

The term “unit dosage form” or “unit dose composition” as used herein refers to a quantity of a compound, such as a drop or a droplet, said quantity being such that one or more predetermined units may be provided as a single therapeutic administration.

As used herein, “treat,” “treatment”, and “treating” refer to the reduction or amelioration of the progression, severity, and/or duration of a given disease resulting from the administration of one or more therapies (including, but not limited to, the administration of an ophthalmic formulation). In certain aspects, the terms refer to the reduction of pain associated with one or more diseases or conditions.

The term “bioavailability” generally refers to the rate and extent to which the active ingredient is absorbed from a drug product and becomes available at the site of action.

The term “ocular bioavailability” as used herein refers to the bioavailability of the active ingredient in an ocular tissue of interest.

The term “wt %” or “weight/volume” as used herein refers to the ratio between two components with respect to volume. For example, a 5 wt % ethanol in water solution would represent a solution comprising 5 g ethanol for every 100 mL water.

The term “mg/mL” as used herein refers to the ratio between a solute, generally an active pharmaceutical ingredient or excipient, and a solvent, generally but not necessarily water. For example, a 50 mg/mL sodium chloride aqueous solution would represent a solution comprising 50 mg sodium chloride for every 1 mL water.

### Pharmaceutical Compositions

Pharmaceutical compositions of this disclosure include an effective amount of a gabapentinoid or a pharmaceutically acceptable salt thereof and one or more pharmaceutically acceptable excipients formulated for ophthalmic administration. In some aspects, the pharmaceutical compositions and methods are useful in the treatment of corneal pain. In some aspects, the corneal pain is acute corneal pain. In some aspects, the corneal pain is chronic corneal pain. In some aspects, the corneal pain is neuropathic corneal pain. In some aspects, the pharmaceutical composition is an ophthalmic formulation comprising a gabapentinoid present at a concentration of from about 0.1 wt % to about 6.5 wt % and one or more pharmaceutically acceptable excipients. In other aspects, the formulation has a viscosity of from about 1 to about 300 cPs and a pH from about 4 to about 8.

In some aspects, the pharmaceutical composition is an ophthalmic formulation consisting essentially of a gabapentinoid present at a concentration of from about 0.1 wt % to about 6.5 wt % and one or more pharmaceutically acceptable excipients. In some aspects the formulation has a viscosity of from about 1 to about 300 cPs. In some aspects, the formulation has a pH from about 4 to about 8.

In some aspects, the gabapentinoid is pregabalin, gabapentin, mirogabalin, gabapentin enacarbil, phenibut, imagabalin, atagabalin, or 4-methylpregabalin.

In some aspects, the pharmaceutical composition is an ophthalmic formulation comprising a gabapentinoid present at a concentration of from about 0.1 wt % to about 6.5 wt %. In some aspects, the pharmaceutical composition is an ophthalmic formulation comprising a gabapentinoid present at a concentration of from about 0.1 wt % to about 0.5 wt %, from about 0.1 wt % to about 1 wt %, from about 0.1 wt % to about 1.5 wt %, from about 0.1 wt % to about 2 wt %, from about 0.1 wt % to about 2.5 wt %, from about 0.1 wt % to about 3 wt %, from about 0.1 wt % to about 3.5 wt %, from about 0.1 wt % to about 4 wt %, from about 0.1 wt % to about 4.5 wt %, from about 0.1 wt % to about 5 wt %, from about 0.1 wt % to about 5.5 wt %, from about 0.1 wt % to about 6 wt %, from about 0.5 wt % to about 1.5 wt %, from about 0.5 wt % to about 2.5 wt %, from about 0.5 wt % to about 3.5 wt %, from about 0.5 wt % to about 4.5 wt %, from about 0.5 wt % to about 5.5 wt %, from about 1.5 wt % to about 2.5 wt %, from about 1.5 wt % to about 3.5 wt %, from about 1.5 wt % to about 4.5 wt %, from about 1.5 wt % to about 5.5 wt %, from about 1.5 wt % to about 6.5 wt %, from about 2.5 wt % to about 3.5 wt %, from about 2.5 wt % to about 4.5 wt %, from about 2.5 wt % to about 5.5 wt %, from about 2.5 wt % to about 6.5 wt %, from about 3.5 wt % to about 4.5 wt %, from about 3.5 wt % to about 5.5 wt %, from about 3.5 wt % to about 6.5 wt %, from about 4.5 wt % to about 5.5 wt %, from about 4.5 wt % to about 6.5 wt %, from about 5.5 wt % to about 6.5 wt %, from about 1 wt % to about 2 wt %, from about 1 wt % to about 3 wt %, from about 1 wt % to about 4 wt %, from about 1 wt % to about 5 wt %, from about 1 wt % to about 6 wt %, from about 2 wt % to about 3 wt %, from about 2 wt % to about 4 wt %, from about 2 wt % to about 5 wt %, from about 2 wt % to about 6 wt %, from about 3 wt % to about 4 wt %, from about 3 wt % to about 5 wt %, from about 3 wt % to about 6 wt %, from about 4 wt % to about 5 wt %, from about 4 wt % to about 6 wt %, or from about 5 wt % to about 6 wt %. In some aspects, the gabapentinoid is pregabalin. In other aspects, the gabapentinoid is gabapentin.

In some aspects, the pharmaceutical composition is an ophthalmic formulation comprising a gabapentinoid present at a concentration of about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, or about 6.5 wt %.

In some aspects, the pharmaceutical composition is an ophthalmic formulation comprising a gabapentinoid present at a concentration of about 0.1 mg/mL to about 6.5 mg/mL. In some aspects, the pharmaceutical composition is an ophthalmic formulation comprising a gabapentinoid present at a concentration of from about 0.1 mg/mL to about 0.5 mg/mL, from about 0.1 mg/mL to about 1 mg/mL, from about 0.1 mg/mL to about 1.5 mg/mL, from about 0.1 mg/mL to about 2 mg/mL, from about 0.1 mg/mL to about 2.5 mg/mL, from about 0.1 mg/mL to about 3 mg/mL, from about 0.1 mg/mL to about 3.5 mg/mL, from about 0.1 mg/mL to about 4 mg/mL, from about 0.1 mg/mL to about 4.5 mg/mL, from about 0.1 mg/mL to about 5 mg/mL, from about 0.1 mg/mL to about 5.5 mg/mL, from about 0.1 mg/mL to about 6 mg/mL, from about 0.5 mg/mL to about 1.5 mg/mL, from about 0.5 mg/mL to about 2.5 mg/mL, from about 0.5 mg/mL to about 3.5 mg/mL, from about 0.5 mg/mL to about 4.5 mg/mL, from about 0.5 mg/mL to about 5.5 mg/mL, from about 1.5 mg/mL to about 2.5 mg/mL, from about 1.5 mg/mL to about 3.5 mg/mL, from about 1.5 mg/mL to about 4.5 mg/mL, from about 1.5 mg/mL to about 5.5 mg/mL, from about 1.5 mg/mL to about 6.5 mg/mL, from about 2.5 mg/mL to about 3.5 mg/mL, from about 2.5 mg/mL to about 4.5 mg/mL, from about 2.5 mg/mL to about 5.5 mg/mL, from about 2.5 mg/mL to about 6.5 mg/mL, from about 3.5 mg/mL to about 4.5 mg/mL, from about 3.5 mg/mL to about 5.5 mg/mL, from about 3.5 mg/mL to about 6.5 mg/mL, from about 4.5 mg/mL to about 5.5 mg/mL, from about 4.5 mg/mL to about 6.5 mg/mL, from about 5.5 mg/mL to about 6.5 mg/mL, from about 1 mg/mL to about 2 mg/mL, from about 1 mg/mL to about 3 mg/mL, from about 1 mg/mL to about 4 mg/mL, from about 1 mg/mL to about 5 mg/mL, from about 1 mg/mL to about 6 mg/mL, from about 2 mg/mL to about 3 mg/mL, from about 2 mg/mL to about 4 mg/mL, from about 2 mg/mL to about 5 mg/mL, from about 2 mg/mL to about 6 mg/mL, from about 3 mg/mL to about 4 mg/mL, from about 3 mg/mL to about 5 mg/mL, from about 3 mg/mL to about 6 mg/mL, from about 4 mg/mL to about 5 mg/mL, from about 4 mg/mL to about 6 mg/mL, or from about 5 mg/mL to about 6 mg/mL. In some aspects, the gabapentinoid is pregabalin. In other aspects, the gabapentinoid is gabapentin.

In some aspects, the pharmaceutical composition is an ophthalmic formulation comprising a gabapentinoid present at a concentration of about 0.1 mg/mL, about 0.5 mg/mL, about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, or about 6.5 mg/mL.

Viscosity is a quality of an ophthalmic formulation that is critical to maintain residence time of the formulation in the eye. It is important that the viscosity of the formulation is maintained over the life of the formulation so that the viscosity is consistent and the delivery and residence time of the active ingredient is maintained. Viscosity agents can be added to ophthalmic formulations to adjust the viscosity of the formulation. In some aspects, the pharmaceutical composition comprises at least one viscosity agent. Non-limiting examples of viscosity agents include polyvinyl alcohol, polyvinylpyrrolidone, hydroxypropylmethylcellulose, poloxamers, carboxymethylcellulose, poly(acrylic acid), hydroxyethylcellulose, hyaluronic acid, and their derivatives.

In some aspects, the pharmaceutical composition has a viscosity of from about 1 cPs to about 300 cPs . In some aspects, the pharmaceutical composition has a viscosity of from about 1 cPs to about 50 cPs, about 1 cPs to about 100 cPs, about 1 cPs to about 150 cPs, about 1 cPs to about 200 cPs, about 1 cPs to about 250 cPs, about 1 cPs to about 300 cPs, about 50 cPs to about 100 cPs, about 50 cPs to about 150 cPs, about 50 cPs to about 200 cPs, about 50 cPs to about 250 cPs, about 50 cPs to about 300 cPs, about 100 cPs to about 150 cPs, about 100 cPs to about 200 cPs, about 100 cPs to about 250 cPs, about 100 cPs to about 300 cPs, about 150 cPs to about 200 cPs, about 150 cPs to about 250 cPs, about 150 cPs to about 300 cPs, about 200 cPs to about 250 cPs, about 200 cPs to about 300 cPs, or about 250 cPs to about 300 cPs,. In some aspects, the pharmaceutical composition has a viscosity of about 1 cPs, about 50 cPs, about 100 cPs, about 150 cPs, about 200 cPs, about 250 cPs, or about 300 cPs.

In some aspects, the pharmaceutical composition has a pH of from about 4 to about 8. In some aspects, the pharmaceutical composition has a pH of from about 4 to about 4.5, from about 4 to about 5, from about 4 to about 5.5, from about 4 to about 6, from about 4 to about 6.5, from about 4 to about 7, from about 4 to about 7.5, from about 4 to about 8, from about 4.5 to about 5, from about 4.5 to about 5.5, from about 4.5 to about 6, from about 4.5 to about 6.5, from about 4.5 to about 7, from about 4.5 to about 7.5, from about 4.5 to about 8, from about 5 to about 5.5, from about 5 to about 6, from about 5 to about 6.5, from about 5 to about 7, from about 5 to about 7.5, from about 5 to about 8, from about 5.5 to about 6, from about 5.5 to about 6.5, from about 5.5 to about 7, from about 5.5 to about 7.5, from about 5.5 to about 8, from about 6 to about 6.5, from about 6 to about 7, from about 6 to about 7.5, from about 6 to about 8, from about 6.5 to about 7, from about 6.5 to about 7.5, from about 6.5 to about 8, from about 7 to about 7.5, from about 7 to about 8, or from about 7.5 to about 8.

In some aspects, the pharmaceutical composition has a pH of about 4, about 4.5, about 5, about 5.5, about 6, about 6.5, about 7, about 7.5, or about 8.

In some aspects, the pharmaceutical composition is an ophthalmic formulation comprising a solution. In some aspects, the composition comprises an aqueous solution, a gel, a semi-solid gel, an ointment, or a suspension. In some aspects, the composition comprises a silicone oil. In some aspects, the composition is a spreadable film. In some aspects, the composition comprises a hydrocarbon or silicone wax. In some aspects, the pharmaceutical composition comprises a co-solvent including but not limited to ethanol, isopropyl alcohol, dimethylsulfoxide, and polyethylene glycol.

In some aspects, the pharmaceutical composition is an ophthalmic formulation comprising a gabapentinoid, wherein the gabapentinoid is in suspension in the composition. If the gabapentinoid is in suspension in the composition, the present composition may include an effective amount of resuspension component effective to facilitate the suspension or resuspension of the gabapentinoid particles in the present compositions. Non-limiting examples of resuspension components include surfactants such as poloxanes, for example, sold under the trademark PLURONIC®, tyloxapol, sarcosinates, polyethoxylated castor oils, other surfactants and mixtures thereof

In some aspects, the pharmaceutical composition comprises one or more pharmaceutically acceptable excipients. In some aspects, the excipient is a preservative, a buffer, a penetration enhancer, a viscosity agent, a tonicity regulator, a chelating agent, a polymer, a lipid, or an ointment base, or a combination of excipients thereof. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

In some aspects, the pharmaceutical composition comprises a preservative. Non-limiting examples of such preservatives include benzalkonium chloride, chlorhexidine, PHMB (polyhexamethylene biguanide), methyl and ethyl parabens, hexetidine, chlorite components, such as stabilized chlorine dioxide, metal chlorites and the like, other ophthalmically acceptable preservatives and the like and mixtures thereof. The concentration of the preservative in the composition is a concentration effective to preserve the composition. In some aspects, the concentration of the preservative is from about 0.00001 wt % to about 0.0001 wt %, from about 0.00001 wt % to about 0.001 wt %, from about 0.00001 wt % to about 0.01 wt %, from about 0.00001 wt % to about 0.1 wt %, from about 0.0001 wt % to about 0.001 wt %, from about 0.0001 wt % to about 0.01 wt %, from about 0.0001 wt % to about 0.1 wt %, from about 0.001 wt % to about 0.01 wt %, from about 0.001 wt % to about 0.1 wt %, or from about 0.01 wt % to about 0.1 wt %.

In some aspects, the concentration of the preservative is about 0.00001 wt %, about 0.0001 wt %, about 0.001 wt %, about 0.01 wt %, or about 0.1 wt %.

In some aspects, the pharmaceutical composition is essentially free of a preservative. In some aspects, the pharmaceutical composition is free of preservatives.

In some aspects, the pharmaceutical composition comprises one or more additional active pharmaceutical ingredients. In some aspects, the second active agent is an antimicrobial agent. Non-limiting examples of antimicrobial agents include gentamycin, tobramycin, paromomycin, kanamycin, neomycin, vancomycin, amikacin, moxifloxacin, gatifloxacin, levofloxacin, gemifloxacin, ciprofloxacin, norfloxacin, ofloxacin, sulfacetamide, sulfadiazine, sulfadimidine, sulfafurazole, sulfisomidine, sulfadoxine, sulfamethoxazole, sulfamoxole, sulfanitran, sulfadimethoxine, sulfamethoxypyridazine, sulfametoxydiazine ,sulfametopyrazine, penicillin G, penicillin V, benzylpenicillins, phenoxymethylpenicillin, dicloxacillin, flucloxacillin, oxacillin, nafcillin, amoxicillin, ticarcillin, piperacillin, ritipenem, ertapenem, doripenem, imipenem and meropenem, cefazolin, cefalexin, cefadroxil, cefapirin, cefaclor, cefotetan, cephamycin, cefprozil, cefuroxime axetil, ceftriaxone, ceftazidime, cefoperazone, cefdinir, cefcapene, cefdaloxime, ceftizoxime, cefmenoxime, cefotaxime, cefpiramide, cefpodoxime, ceftibuten, cefditoren, cefepime, ceftaroline fosamil, ceftolozane, ceftobiprole, ceftiofur, cefquinome and cefovecin, aztreonam, sulbactam, tazobactam, clavulanic acid, avibactam, or cyclosporine, or combinations thereof. In some aspects, the concentration of the second active agent is from about 1 wt % to about 2 wt %, from about 1 wt % to about 3 wt %, from about 1 wt % to about 4 wt %, from about 1 wt % to about 5 wt %, from about 2 wt % to about 3 wt %, from about 2 wt % to about 4 wt %, from about 2 wt % to about 5 wt %, from about 3 wt % to about 4 wt %, from about 3 wt % to about 5 wt %, or from about 4 wt % to about 5 wt %. In some aspects, the concentration of the second active agent is about 1 wt %, about 2 wt %, about 3 wt %, about 4 wt %, or about 5 wt %.

In some aspects, the pharmaceutical composition comprises one or more buffers in an amount effective to control and/or maintain the pH of the composition. The buffer may be chosen from those that are conventional and well known in the ophthalmic art. Non-limiting examples of such buffers include acetate buffers, citrate buffers, phosphate buffers, borate buffers, borate-polyol buffers, carbonate buffers, organic buffers, amino acid buffers, TRIS buffers, and combinations thereof. The amount of buffer employed preferably is sufficient to maintain the pH of the composition in a range of from about 5 to about 5.5, from about 5 to about 6, from about 5 to about 6.5, from about 5 to about 7, from about 5 to about 7.5, from about 5 to about 8, from about 5.5 to about 6, from about 5.5 to about 6.5, from about 5.5 to about 7, from about 5.5 to about 7.5, from about 5.5 to about 8, from about 6 to about 6.5, from about 6 to about 7, from about 6 to about 7.5, from about 6 to about 8, from about 6.5 to about 7, from about 6.5 to about 7.5, from about 6.5 to about 8, from about 7 to about 7.5, or from about 7.5 to about 8.

In some aspects, the pharmaceutical composition comprises at least one tonicity regulator in an amount effective to control the tonicity or osmolality of the composition. Non-limiting examples of tonicity components include salts, particularly sodium chloride, potassium chloride, glycerin, mannitol, dextrose, and other sugar alcohols, and other suitable ophthalmically acceptably tonicity components and mixtures thereof.

In some aspects, the pharmaceutical composition comprises at least one penetration enhancer. Non-limiting examples of penetration enhancers include surfactants, certain organic solvents such as benzalkonium chloride, dimethylsulfoxide and other sulfoxides, dimethylacetamide and pyrrolidone, certain amides of heterocyclic amines, glycols (e.g. propyleneglycol), propylene carbonate, oleic acid, alkylamines and derivatives, various cationic, anionic and nonionic surfactants, amphoteric surfactants and the like.

In some aspects, the pharmaceutical composition comprises at least one chelating agent. In one aspect, the chelating agent is disodium edetate but others can be used instead of or in combination with this.

In some aspects, the pharmaceutical composition has an osmolality of from about 260 to about 365 mOsm/kg. In some aspects, the pharmaceutical composition has an osmolality of from about 285 mOsm/kg to about 295 mOsm/kg, from about 285 mOsm/kg to about 305 mOsm/kg, from about 285 mOsm/kg to about 315 mOsm/kg, from about 285 mOsm/kg to about 325 mOsm/kg, from about 285 mOsm/kg to about 335 mOsm/kg, from about 285 mOsm/kg to about 345 mOsm/kg, from about 285 mOsm/kg to about 355 mOsm/kg, from about 285 mOsm/kg to about 365 mOsm/kg, from about 295 mOsm/kg to about 305 mOsm/kg, from about 295 mOsm/kg to about 315 mOsm/kg, from about 295 mOsm/kg to about 325 mOsm/kg, from about 295 mOsm/kg to about 335 mOsm/kg, from about 295 mOsm/kg to about 345 mOsm/kg, from about 295 mOsm/kg to about 355 mOsm/kg, from about 295 mOsm/kg to about 365 mOsm/kg, from about 305 mOsm/kg to about 315 mOsm/kg, from about 305 mOsm/kg to about 325 mOsm/kg, from about 305 mOsm/kg to about 335 mOsm/kg, from about 305 mOsm/kg to about 345 mOsm/kg, from about 305 mOsm/kg to about 355 mOsm/kg, from about 305 mOsm/kg to about 365 mOsm/kg, from about 315 mOsm/kg to about 325 mOsm/kg, from about 315 mOsm/kg to about 335 mOsm/kg, from about 315 mOsm/kg to about 345 mOsm/kg, from about 315 mOsm/kg to about 355 mOsm/kg, from about 315 mOsm/kg to about 365 mOsm/kg, from about 325 mOsm/kg to about 335 mOsm/kg, from about 325 mOsm/kg to about 345 mOsm/kg, from about 325 mOsm/kg to about 355 mOsm/kg, from about 325 mOsm/kg to about 365 mOsm/kg, from about 335 mOsm/kg to about 345 mOsm/kg, from about 335 mOsm/kg to about 355 mOsm/kg, from about 335 mOsm/kg to about 365 mOsm/kg, from about 345 mOsm/kg to about 355 mOsm/kg, from about 345 mOsm/kg to about 365 mOsm/kg, from about 355 mOsm/kg to about 365 mOsm/kg, from about 260 mOsm/kg to about 265 mOsm/kg, from about 260 mOsm/kg to about 275 mOsm/kg, from about 260 mOsm/kg to about 285 mOsm/kg, from about 260 mOsm/kg to about 295 mOsm/kg, from about 260 mOsm/kg to about 305 mOsm/kg, from about 260 mOsm/kg to about 315 mOsm/kg, from about 260 mOsm/kg to about 325 mOsm/kg, from about 260 mOsm/kg to about 335 mOsm/kg, from about 260 mOsm/kg to about 345 mOsm/kg, from about 260 mOsm/kg to about 355 mOsm/kg, from about 260 mOsm/kg to about 365 mOsm/kg, from about 265 mOsm/kg to about 275 mOsm/kg, from about 265 mOsm/kg to about 285 mOsm/kg, from about 265 mOsm/kg to about 295 mOsm/kg, from about 265 mOsm/kg to about 305 mOsm/kg, from about 265 mOsm/kg to about 315 mOsm/kg, from about 265 mOsm/kg to about 325 mOsm/kg, from about 265 mOsm/kg to about 335 mOsm/kg, from about 265 mOsm/kg to about 345 mOsm/kg, from about 265 mOsm/kg to about 355 mOsm/kg, from about 265 mOsm/kg to about 365 mOsm/kg, from about 275 mOsm/kg to about 285 mOsm/kg, from about 275 mOsm/kg to about 295 mOsm/kg, from about 275 mOsm/kg to about 305 mOsm/kg, from about 275 mOsm/kg to about 315 mOsm/kg, from about 275 mOsm/kg to about 325 mOsm/kg, from about 275 mOsm/kg to about 335 mOsm/kg, from about 275 mOsm/kg to about 345 mOsm/kg, from about 275 mOsm/kg to about 355 mOsm/kg, from about 275 mOsm/kg to about 365 mOsm/kg.

In some aspects, the pharmaceutical composition has an osmolality of about about 260 mOsm/kg, about 265 mOsm/kg, about 275 mOsm/kg, about 285 mOsm/kg, about 295 mOsm/kg, about 305 mOsm/kg, about 315 mOsm/kg, about 325 mOsm/kg, about 335 mOsm/kg, about 345 mOsm/kg, about 355 mOsm/kg, or about 365 mOsm/kg

In some aspects, the pharmaceutical composition is isotonic. In some aspects, the pharmaceutical composition is hypotonic. In some aspects, the pharmaceutical composition is hypertonic.

In some aspects, the pharmaceutical composition comprises a pharmaceutically acceptable polymer. Non-limiting examples of polymers include polyesters, polycaprolactone, polyanhydrides, polyvinylpyrrolidone, polyethylene glycol, polysaccharides, poly(D,L-lactic-co-glycolic acid, polylactic acid, polyurethane, and polyglycolic acid.

In some aspects, the pharmaceutical composition comprises a pharmaceutically acceptable lipid. Non-limiting examples of lipids include fatty acids, glycerolipids such as monoglycerides, diglycerides, and triglycerides, phospholipids, sphingolipids, sterol lipids, prenol lipids, saccharolipids, polyketides, and mixtures thereof.

In some aspects, the pharmaceutical composition comprises a pharmaceutically acceptable ointment base. Non-limiting examples of ointment bases include mineral oil, white petrolatum, anhydrous lanolin, and mixtures thereof.

In some aspects, the pharmaceutical composition is formulated for a single dose. In some aspects, the pharmaceutical composition is formulated for multiple doses. In some aspects, the pharmaceutical composition is packaged in an aseptic manner. In some aspects, the pharmaceutical composition is sterile.

In some aspects, the pharmaceutical composition is stable. In some aspects, the pharmaceutical composition is stable for at least 1 month at 25° C. In some aspects, the pharmaceutical composition is stable for at least 3 months at 25° C. In some aspects, the pharmaceutical composition is stable for at least 6 months at 25° C. In some aspects, the pharmaceutical composition is stable for at least 12 months at 25° C. In some aspects, the pharmaceutical composition is stable for at least 18 months at 25° C.

### Methods of Treatment

The present disclosure also provides a method for treating corneal pain in a subject, the method comprising administration to at least one eye of a subject in need thereof a therapeutically effective amount of a pharmaceutically acceptable ophthalmic composition, the composition comprising a gabapentinoid present at a concentration of from about 0.1 to about 6.5 wt %. In some aspects, the gabapentinoid is pregabalin. In other aspects, the gabapentinoid is gabapentin.

In some aspects, the corneal pain is acute corneal pain. In some aspects, the corneal pain is chronic corneal pain. In some aspects, the corneal pain is neuropathic corneal pain. In some aspects, the pain is caused by corneal nerve damage, LASIK surgery, cataract surgery, or other eye surgeries, chronic dry eye disease, radiation therapy, extended contact lens wear, toxicity from preservatives, shingles, diabetes, trigeminal neuralgia, abnormal healing, corneal abrasions, physical or chemical injury, or any systemic condition that can lead to nerve damage.

In some aspects, the administering is performed by delivery of drops to the ocular surface. In some aspects, the administering is performed by intravitreal or intraocular injection.

In some aspects, the subject is a mammal. In some aspects, the pharmaceutical composition is suitable for veterinary use. In some aspects, the subject is a human.

In some aspects, the pharmaceutical composition is co-administered with a second pharmaceutical composition, wherein the second composition comprises a non-steroidal anti-inflammatory drug (NSAID). Non-limiting examples of NSAIDs include acetylsalicylic acid, acetaminophen, ibuprofen, naproxen, nepafenac, bromfenac, diclofenac, flurbiprofen, ketoprofen, ketorolac, indomethacin, and sulindac.

In some aspects, the pharmaceutical composition is co-administered with a second pharmaceutical composition, wherein the second composition comprises a local anesthetic. Non-limiting examples of local anesthetics include ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, or a pharmaceutically acceptable salt thereof, or a mixture thereof.

In some aspects, the pharmaceutical composition is co-administered with a second pharmaceutical composition, wherein the second composition comprises an immune suppressant. Non-limiting examples of immune suppressants include cyclosporine, tacrolimus, sirolimus, everolimus, and zotarolimus.

In some aspects, the pharmaceutical composition is co-administered with a second pharmaceutical composition, wherein the second composition comprises a steroid. Non-limiting examples of steroids include corticosteroids, such as cortisone, prednisolone, flurometholone, dexamethasone, medrysone, loteprednol, fluazacort, hydrocortisone, prednisone, betamethasone, prednisone, methylprednisolone, riameinolone hexacatonide, paramethasone acetate, diflorasone, fluocinonide, fluocinolone, triamcinolone, derivatives thereof, and mixtures thereof.

In some aspects, the pharmaceutical composition is co-administered with a second pharmaceutical composition, wherein the second composition comprises an antimicrobial agent. Non-limiting examples of antimicrobial agents are listed in the previous section.

In some aspects, use of one of the disclosed methods result in an increase in ocular bioavailability of a gabapentinoid in a subject compared to oral administration of the same gabapentinoid. In general, an increase in ocular bioavailability may be calculated by taking the difference in the AUC measured in an ocular tissue of interest (e.g., in aqueous humor) between those of a test composition and a control composition, and dividing the difference by the bioavailability of the control composition. A test composition may include an ophthalmic formulation comprising a gabapentinoid that is administered topically to the eye of a subject. A control composition may include an oral formulation of the same gabapentinoid that is administered orally to a subject. Ocular bioavailability of the gabapentinoid may be measured in an appropriate animal model (e.g. in a New Zealand white rabbit model). One method of determining the ocular concentration of the gabapentinoid involves dissecting of the eye of the animal model to isolate tissues of interest. The concentration of the gabapentinoid in the tissues of interest is then determined by HPLC or LC/MS analysis. In some aspects, the concentration of the gabapentinoid may be measured in the eye of the subject directly or indirectly (e.g., taking a sample of fluid, such as vitreous humor, from an eye of the subject).

In some aspects, the concentration of the pharmaceutical agent in an ocular tissue and/or fluid may increase due to, at least in part, administering to the subject an ophthalmic formulation comprising a gabapentinoid, compared to oral administration of an oral formulation comprising the same gabapentinoid. In some aspects, the method of topical administration of an ophthalmic formulation comprising a gabapentinoid described herein increases the concentration of the gabapentinoid in an ocular tissue and/or fluid by about 10%, about 20%, about 30%, about 40%, about 50%, about 60%, about 70%, about 80%, about 90%, about 100%, about 200%, about 300%, about 400%, about 500%, or about 10 fold, about 100 fold, or about 1000 fold, compared to oral administration of an oral formulation comprising the same gabapentinoid. In some aspects, the concentration of the gabapentinoid increases at a tissue and/or fluid in the posterior chamber of the eye. In some aspects, the concentration of the gabapentinoid increases at a tissue and/or fluid in the anterior chamber of the eye.

In some aspects, the methods or compositions described herein may be used to treat, diagnose, prevent, or manage an ocular condition, i.e., a disease, ailment, or condition that affects or involves the eye or one or more of the parts or regions of the eye. Broadly speaking, the eye includes the eyeball and the tissues and fluids which constitute the eyeball, the periocular muscles (such as the oblique and rectus muscles) and the portion of the optic nerve which is within or adjacent to the eyeball.

In some aspects, the methods or compositions described herein may be used to target and/or treat the all or portions of the cornea of a subject. The cornea refers to the convex, transparent anterior part of the eye, comprising one sixth of the outermost tunic of the eye bulb. It allows light to pass through it to the lens. The cornea is a fibrous structure with five layers: the anterior corneal epithelium, continuous with that of the conjunctiva; the anterior limiting layer (Bowman's membrane); the substantial propria; the posterior limiting layer (Descemet's membrane); and the endothelium of the anterior chamber (keratoderma). It is dense, uniform in thickness, and nonvascular, and it projects like a dome beyond the sclera, which forms the other five sixths of the eye's outermost tunic. The degree of corneal curvature varies among different individuals and in the same person at different ages; the curvature is more pronounced in youth than in advanced age.

The efficacy of the methods or compositions of the present disclosure can be evaluated using a variety of ocular pain questionnaires to assess the level of pain experienced by a subject. Non-limiting examples of ocular pain questionnaires include Ocular Pain Assessment Survey, visual analog scale (VAS), numerical rating scale (NRS), Wong-Baker FACES ° Pain Rating Scale, Eye Sensation Scale and any general pain questionnaires that are appropriate for assessment of ocular pain. In some aspects, topical administration of an ophthalmic formulation of a gabapentinoid to at least one eye of a subject results in a decrease in pain of from about 10% to about 30%, from about 10% to about 50%, from about 10% to about 70%, from about 10% to about 90%, from about 10% to about 100%, from about 30% to about 50%, from about 30% to about 70%, from about 30% to about 90%, from about 30% to about 100%, from about 50% to about 70%, from about 50% to about 90%, from about 50% to about 100%, from about 70% to about 90%, from about 70% to about 100%, or from about 90% to about 100%, compared to oral administration of the same gabapentinoid, as measured using one of the aforementioned ocular pain questionnaires. In some aspects, topical administration of an ophthalmic formulation of a gabapentinoid to at least one eye of a subject results in a decrease in pain of about 10%, about 30%, about 50%, about 70%, about 90%, or about 100%, compared to oral administration of the same gabapentinoid, as measured using one of the aforementioned ocular pain questionnaires.

In some aspects, topical administration of an ophthalmic formulation comprising a gabapentinoid results in decreased side effects compared to oral administration of the same gabapentinoid. Non-limiting examples of side effects include angioedema, hypersensitivity reactions, suicidal thoughts or behavior, respiratory depression, dizziness, somnolence, peripheral edema, blurred vision, weight gain, and difficulty with concentration or attention, or combinations thereof

In some aspects, a subject having or at risk of having corneal pain is administered any of the pharmaceutical compositions described herein. In some aspects, the corneal pain is acute corneal pain. In some aspects, the corneal pain is chronic corneal pain. In some aspects, the corneal pain is neuropathic corneal pain. In some aspects, a therapeutically effective dose comprises a gabapentinoid and can be one drop per eye. In some aspects, the pharmaceutical compositions are administered at a constant, therapeutically effective dose from the onset of therapy. In some aspects, the dose comprises two drops of about 0.035 g/drop of an ophthalmic solution comprising a gabapentinoid. In some aspects, the gabapentinoid is pregabalin. In other aspects, the gabapentinoid is gabapentin.

In some aspects, the total dose of the gabapentinoid is from about 0.10 mg to about 0.35 mg, from about 0.10 mg to about 0.70 mg, from about 0.10 mg to about 1.05 mg, from about 0.10 mg to about 1.40 mg, from about 0.10 mg to about 1.75 mg, from about 0.10 mg to about 2.10 mg, from about 0.10 mg to about 2.45 mg, from about 0.10 mg to about 2.80 mg, from about 0.10 mg to about 3.10 mg, from about 0.35 mg to about 0.70 mg, from about 0.35 mg to about 1.05 mg, from about 0.35 mg to about 1.40 mg, from about 0.35 mg to about 1.75 mg, from about 0.35 mg to about 2.10 mg, from about 0.35 mg to about 2.45 mg, from about 0.35 mg to about 2.80 mg, from about 0.35 mg to about 3.10 mg, from about 0.70 mg to about 1.05 mg, from about 0.70 mg to about 1.40 mg, from about 0.70 mg to about 1.75 mg, from about 0.70 mg to about 2.10 mg, from about 0.70 mg to about 2.45 mg, from about 0.70 mg to about 2.80 mg, from about 0.70 mg to about 3.10 mg, from about 1.05 mg to about 1.40 mg, from about 1.05 mg to about 1.75 mg, from about 1.05 mg to about 2.10 mg, from about 1.05 mg to about 2.45 mg, from about 1.05 mg to about 2.80 mg, from about 1.05 mg to about 3.10 mg, from about 1.40 mg to about 1.75 mg, from about 1.40 mg to about 2.10 mg, from about 1.40 mg to about 2.45 mg, from about 1.40 mg to about 2.80 mg, from about 1.40 mg to about 3.10 mg, from about 1.75 mg to about 2.10 mg, from about 1.75 mg to about 2.45 mg, from about 1.75 mg to about 2.80 mg, from about 1.75 mg to about 3.10 mg, from about 2.10 mg to about 2.45 mg, from about 2.10 mg to about 2.80 mg, from about 2.10 mg to about 3.10 mg, from about 2.45 mg to about 2.80 mg, from about 2.45 mg to about 3.10 mg, or from about 2.80 mg to about 3.10 mg.

In some aspects, the total dose of the gabapentinoid is about 0.10 mg, about 0.35 mg, about 0.70 mg, about 1.05 mg, about 1.40 mg, about 1.75 mg, about 2.10 mg, about 2.45 mg, about 2.80 mg, or about 3.10 mg.

In some aspects, the pharmaceutical composition is administered once daily to one or both eyes. In some aspects, the composition is administered in the morning. In some aspects, the composition is administered in the afternoon. In some aspects, the composition is administered in the evening. In some aspects, the composition is administered more than 4 hours before bedtime. In some aspects, the composition is administered on one or more consecutive days. In some aspects, the composition is administered multiple times per day. In some aspects, the composition is administered 2 times per day, 3 times per day, 4 times per day, 5 times per day, 6 times per day, 7 times per day, 8 times per day, 9 times per day, 10 times per day, 11 times per day or 12 times per day. In some aspects, the composition is administered 2 to 64 times per day, 2 to 24 times per day, 2 to 16 times per day, 2 to 12 times per day, 2 to 8 times per day, 2 to 4 times per day, 8 to 16 times per day, or 4 to 12 times per day. In some aspects, the composition is administered once per hour. In some aspects, the composition is administered once about every 15 minutes. In some aspects, the composition is administered once about every 30 minutes. In some aspects, the composition is administered as needed. In some aspects, a single drop is administered to each eye during each dose administration. In some aspects, a single drop is administered to only one eye during each dose administration.

### EXAMPLES

### Examples 1-6

TABLE 1
provides formulation details of six exemplary pregabalin ophthalmic
formulations (Examples 1-6) of the present disclosure.

| | Example 1 | Example 2 | Example 3 | Example 4 | Example 5 | Example 6 |
|---|---|---|---|---|---|---|
| Pregabalin | 0.1-6 wt % | 0.1-6 wt % | 0.1-6 wt % | 0.1-6 wt % | 0.1-6 wt % | 0.1-6 wt % |
| Sodium | 0.6-1 wt % | 0.6-1 wt % | 0.6-1 wt % | — | — | 0.6-1 wt % |
| chloride | | | | | | |
| Benzalkonium | — | — | 0.001-0.02 | — | — | — |
| chloride | | | wt % | | | |
| Phosphate | — | — | 100 mM, | 100 mM, | — | 100 mM, |
| buffer | | | pH 7 | pH 7.5 | | pH 7 |
| Hydroxypropyl- | — | 0.5-1 wt % | 0.5-1 wt % | | — | 0.5-1 wt % |
| methylcellulose | | | | | | |

### Examples 7-12

TABLE 2
provides formulation details of six exemplary gabapentin ophthalmic
formulations (Examples 7-12) of the present disclosure.

| | Example 1 | Example 2 | Example 3 | Example 4 | Example 5 | Example 6 |
|---|---|---|---|---|---|---|
| Gabapentin | 0.1-6 wt % | 0.1-6 wt % | 0.1-6 wt % | 0.1-6 wt % | 0.1-6 wt % | 0.1-6 wt % |
| Sodium | 0.6-1 wt % | 0.6-1 wt % | 0.6-1 wt % | — | — | 0.6-1 wt % |
| chloride | | | | | | |
| Benzalkonium | — | — | 0.001-0.02 | — | — | — |
| chloride | | | wt % | | | |
| Phosphate | — | — | 100 mM, | 100 mM, | — | 100 mM, |
| buffer | | | pH 7 | pH 7.5 | | pH 7 |
| Hydroxypropyl- | — | 0.5-1 wt % | 0.5-1 wt % | | — | 0.5-1 wt % |
| methylcellulose | | | | | | |

### Example 13: Method of Preparing the Ophthalmic Formulations of Examples 1-12

A general procedure of preparing an ophthalmic formulation of the present disclosure is provided as follows.

If using buffer, prepare and adjust to proper pH.

Add appropriate quantities of sodium chloride, benzalkonium chloride, and hydroxypropylmethylcellulose to buffer solution or pure water and mix well to dissolve.

Add appropriate quantity of pregabalin or gabapentin and mix well to dissolve.

Under a laminar flow hood, aseptically filter solution through a 0.22-micron filter needle into sterile packaging and properly seal packaging.

### Example 14

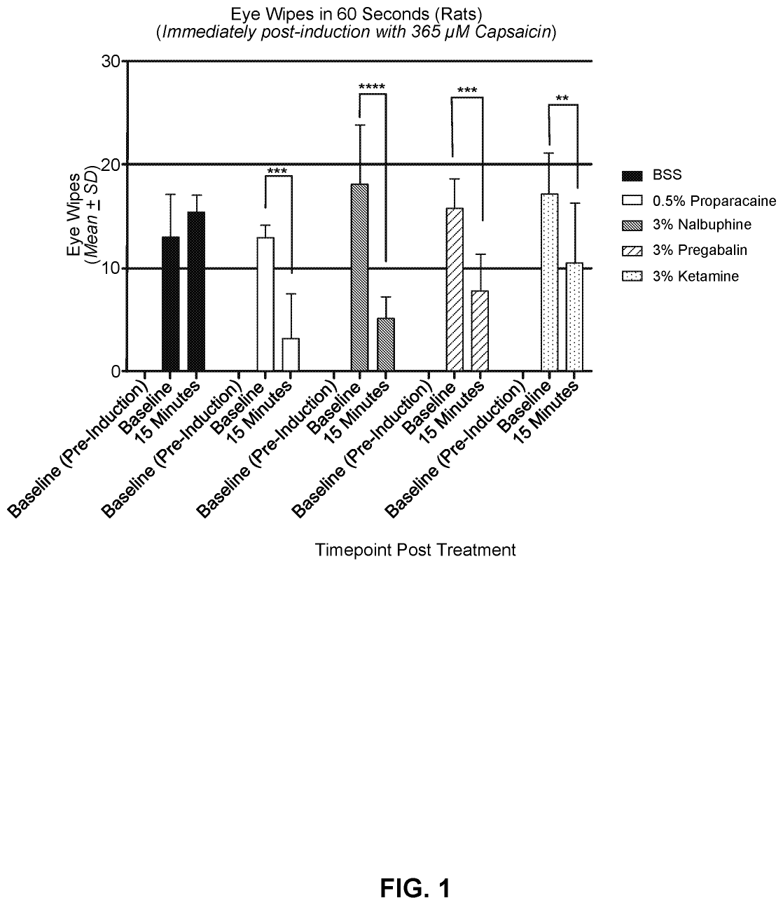

Test ophthalmic formulations were administered to rats with post-surgical corneal injuries to test the efficacy of the formulations in treating corneal pain. A negative control was established by treating all animals with artificial tears (AT) and counting ipsilateral paw wipes for 60 seconds (FIG. 1, Baseline (Pre-induction)). 20 minutes later, a positive control was established by treating all animals with 40 μL 0.1% capsaicin and counting ipsilateral paw wipes for 60 seconds (FIG. 1, Baseline). Eyes were then flushed with balanced salt solution (BSS) and AT. 20 minutes later, all animals were treated with the test ophthalmic formulation (0.5% proparacaine (positive control), 3% nalbuphine, 3% pregabalin, or 3% ketamine). 15 minutes later, all animals were treated with 40 μL 0.1% capsaicin, and ipsilateral paw wipes were counted for 60 seconds (FIG. 1, 15 Minutes). As shown in FIG. 1, nalbuphine and pregabalin resulted in a significant decrease in eye wipes, while ketamine resulted in a borderline response.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What Is Claimed Is:

1. An ophthalmic formulation comprising a gabapentinoid present at a concentration of from about 0.1 to about 6.5 wt % and one or more pharmaceutically acceptable excipients, wherein the formulation has a viscosity of from about 1 to about 300 cPs and a pH from about 5 to about 8.

2. The formulation of claim 1, wherein the gabapentinoid is pregabalin or gabapentin.

3. The formulation of claim 1 or 2, wherein the concentration of the gabapentinoid is from about 0.1 wt % to about 5 wt %.

4. The formulation of claim 3, wherein the concentration of the gabapentinoid is from about 2 wt % to about 4 wt %.

5. The formulation of claim 4, wherein the concentration of the gabapentinoid is about 2 wt %.

6. The formulation of claim 4, wherein the concentration of the gabapentinoid is about 3 wt %. 7 The formulation of claim 4, wherein the concentration of the gabapentinoid is about 4 wt %.

8. The formulation of any one of claims 1-7, wherein the viscosity is from about 5 to about 50 cPs.

9. The formulation of any one of claims 1-8, wherein the pH is from about 6.5 to about 7.5.

10. The formulation of any one of claims 1-9, wherein the formulation is an aqueous solution.

11. The formulation of any one of claims 1-9, wherein the formulation is a suspension.

12. The formulation of any one of claims 1-9, wherein the formulation is a semi-solid gel.

13. The formulation of any one of claims 1-9, wherein the formulation is an ointment.

14. The formulation of any one of claims 1-13, wherein the one or more pharmaceutically acceptable excipients are selected from the group consisting of preservatives, buffers, penetration enhancers, viscosity agents, tonicity regulators, chelating agents, polymers, lipids, ointment bases, and resuspension components, or combinations thereof.

15. The formulation of any one of claims 1-14, wherein the formulation further comprises an antimicrobial agent.

16. The formulation of any one of claims 1-15, wherein the osmolality of the formulation is from about 260 to about 365 mOsm/kg.

17. The formulation of any one of claims 1-16, wherein the formulation is isotonic.

18. The formulation of any one of claims 1-16, wherein the formulation is hypotonic.

19. The formulation of any one of claims 1-16, wherein the formulation is hypertonic.

20. The formulation of any one of claims 1-19, wherein the formulation is packaged in an aseptic manner.

21. The formulation of any one of claims 1-20, wherein the formulation is packaged in a single-dose container.

22. The formulation of any one of claims 1-20, wherein the formulation is packaged in a multiple-dose container.

23. A method of treating corneal pain in a subject, comprising administering to the subject a therapeutically effective amount of the composition of any one of claims 1-22.

24. The method of claim 23, wherein the corneal pain is acute corneal pain.

25. The method of claim 23, wherein the corneal pain is chronic corneal pain.

26. The method of any one of claims 23-25, wherein the subject is a human.

27. The method of any one of claims 23-26, wherein the composition is co-administered with a second pharmaceutical composition.

28. The method of claim 27, wherein the second pharmaceutical composition contains an active agent selected from the group consisting of non-steroidal anti-inflammatory drugs, local anesthetics, antimicrobial agents, immune suppressants, and steroids.

29. The method of any one of claims 23-28, wherein the ocular bioavailability of the gabapentinoid is improved by from about 5% to about 50% compared to oral administration of the same gabapentinoid.

30. The method of any one of claims 23-29, wherein the administering is into the cornea, the lower eyelid, an eye surface, or an ophthalmic tissue of at least one eye of the subject.

31. The method of any one of claims 23-30, wherein the administering is performed by intravitreal or intraocular injection.

32. The method of any one of claims 23-31, wherein the intensity of corneal pain in the subject decreases by from about 5 to about 50% as measured by an ocular pain questionnaire compared to treatment with oral administration of the same gabapentinoid or no treatment.

33. The method of claim 32, wherein the ocular pain questionnaire is selected from the group consisting of Ocular Pain Assessment Survey, visual analog scale (VAS), numerical rating scale (NRS), Wong-Baker FACES® Pain Rating Scale, and Eye Sensation Scale, or combination thereof.

34. The method of any one of claims 23-33, wherein the subject has reduced side effects compared to oral administration of the same gabapentinoid, wherein the side effects are selected from the group consisting of angioedema, hypersensitivity reactions, suicidal thoughts or behavior, respiratory depression, dizziness, somnolence, peripheral edema, blurred vision, weight gain, and difficulty with concentration and attention, or combinations thereof
